(19) United States
(12) Patent Application Publication
(10) Pub. No.: US 2009/0192514 A1
(43) Pub. Date: Jul. 30, 2009

(54) IMPLANTABLE DISTRACTION OSTEOGENESIS DEVICE AND METHODS OF USING SAME

(76) Inventors: Stephen E. Feinberg, Ann Arbor, MI (US); Ann Marie Sastry, Ann Arbor, MI (US); Myoungdo Chung, Ann Arbor, MI (US)

Correspondence Address: BUTZEL LONG;IP DOCKETING DEPT, 350 SOUTH MAIN STREET, SUITE 300, ANN ARBOR, MI, 48104, US

(21) Appl. No.: 12/248,448

(22) Filed: Oct. 9, 2008

Related U.S. Application Data
(60) Provisional application No. 60/978,472, filed on Oct. 9, 2007.

(51) Int. Cl. A61B 17/58

(52) U.S. Cl. 606/90

(57) ABSTRACT

An osteogenesis distraction device for securement to separate bone segments in the body of a patient and operative to generate new bone between said bone segments by the gradual application of a distraction force, the device comprising attachment members securable to separate bone segments and moveable in relation to each other to generate a distraction force, a power source for powering relative movement of the attachment members, and wherein the entire device, including the attachment members and power source, is dimensioned for implantation entirely subdermally in the body of a patient.

### CROSS-REFERENCE TO RELATED APPLICATIONS

This application is related to, and claims the benefit of priority from, U.S. Provisional Patent Application Ser. No. 60/978,472, filed Oct. 9, 2007, the disclosure of which application is incorporated herein by reference in its entirety.

### FIELD OF THE INVENTION

The present invention pertains generally to distraction osteogenesis and, more particularly, to an osteogenesis distraction device comprising attachment members securable to separate bone segments and moveable in relation to each other to generate a distraction force, a power source for powering relative movement of the attachment members, and wherein the entire device, including the attachment members and power source, is dimensioned for implantation entirely subdermally in the body of a patient.

### BACKGROUND OF THE INVENTION

Distraction osteogenesis is a method of generating new bone in a gap between two bone segments, by the gradual application of tensile stress across the bone gap (Swennen G, Schliephake H, Dempf R, Schierle H, Malevez C.: Craniofacial distraction osteogenesis: a review of the literature. Part I: clinical studies, Int J Oral & Maxillofac Surg 30:89-103, 2001). The clinical technique was first applied to craniofacial implications in 1992 by McCarthy et al.; most subsequent research has focused on developing more effective distraction via empirical examination with a variety of clinical parameters such as latency period, distraction rate, and distraction frequency (Swennen G, Schliephake H, Dempf R, Schierle H, Malevez C.: Craniofacial distraction osteogenesis: a review of the literature. Part I: clinical studies, Int J Oral & Maxillofac Surg 30:89-103, 2001). The main criticisms of external apparatuses are unsightly scars and/or injuries to the facial nerve resulting from the transcutaneous pins, and thus lack of acceptance by the patients (Schmelzeisen R, Neumann G, Von der Fecht R.: Distraction osteogenesis in the mandible with a motor-driven plate: a preliminary animal study, Brit J of Oral Maxil Surg 34:375-378, 1996. Overcoming these limitations, internal or intra-oral distraction devices have become the most common clinical apparatus in craniofacial distraction osteogenesis (Swennen G, Schliephake H, Dempf R, Schierle H, Malevez C.: Craniofacial distraction osteogenesis: a review of the literature. Part I: clinical studies, Int J Oral & Maxillofac Surg 30:89-103, 2001). In both external and internal devices, however, the actuation of distraction process relies upon manual length adjustment under patients' compliance, introducing inconvenience and potential error in the procedure. More importantly, continuous distraction by application of a low strain magnitude with multiple steps, and leading to greater osteogenic activity (Ilizarov G A.: The tension-stress effect on the genesis and growth of tissues: II. The influence of the rate and frequency of distraction. Clin Orthop 239:263-85, 1989; Kessler P, Neukam F W, Wilffang J.: Effects of distraction forces and frequency of distraction on bony regeneration. Br J Oral Maxillofac Surg 43:392-398, 2005), is restricted by the manual operation protocol, which limits the distraction frequency under 2-4 times per day.

Kessler et al. (2005) showed that continuous osteodistraction resulted in intramembraneous regeneration of bone, whereas intermittent osteodistraction (used by all present distraction devices) caused chondroid ossification in the regenerate of bone. In addition, continuous osteodistraction caused speedier regeneration and distraction forces, maximum pressure peaks and mean distraction force for maximum extension, respectively, were lower (mean value 1.0 N/mm² and 28.3 N, respectively) than with intermittent distraction (mean value 2.7 N/mm² and 76.3 N, respectively). These critical limitations of intermittent force application with both internal and external procedures motivate the development of new devices for distraction osteogenesis, which are termed “continuous automatic distracters (CAD).”

Continuous distraction has shown definitive advantages over intermittent distraction. For example, Kessler et al. (2005) demonstrated a faster rate of regeneration with less force application. In discontinuous distraction, a rate of 1 mm/day is the most successful, and the most frequently used parameter in previous experimental and clinical studies (Swennen et al., 2001). Those empirical data have shown lower rates of distraction tend to cause mechanical problems (pin loosening, breakage) in devices, while higher distraction rates lead to premature ossification of the callus during distraction osteogenesis (Meyer et al., 2004).

The present invention provides an implantable device which overcomes the problems of prior art devices, both intra-oral and external, and in which the rate of distraction, whether intermittent or continuous in nature, can be attained with a high degree of precision.

### SUMMARY OF THE INVENTION

The present invention addresses the problems of prior art osteogenesis distraction devices, and encompasses other features and advantages, by providing an osteogenesis distraction method and device. The method comprises the steps of providing an osteogenesis distraction device for securement to separate bone segments in the body of a patient and operative to generate new bone between said bone segments by the gradual application of a distraction force, the device comprising attachment members securable to separate bone segments and moveable in relation to each other to generate a distraction force, a power source for powering relative movement of the attachment members; and implanting the entire osteogenesis distraction device, including the attachment members and power source, subdermally in the body of a patient.

In implementation of the foregoing method, there is disclosed herein an osteogenesis distraction device for securement to separate bone segments in the body of a patient and operative to generate new bone between the bone segments by the gradual application of a distraction force. The device comprises attachment members securable to separate bone segments and moveable in relation to each other to generate a distraction force, and a power source for powering relative movement of the attachment members. The entire device, including the attachment members and power source, is dimensioned for implantation entirely subdermally in the body of a patient.

According to one feature of the invention, movement of the attachment members is effected by an actuator operable to generate a distraction force between the attachment members. The actuator may, per one aspect of the invention, comprise an electric motor operatively connected to a rotary-to-linear mechanism. The electric motor may comprise a DC micromotor, for example.

Per another feature, the rotary-to-linear mechanism is a lead screw operatively connected to one of the attachment members.

Per still another feature, the electric motor is operatively coupled to a transmission. The transmission may, according to one embodiment, comprise a planetary gearhead having a high reduction ratio (e.g., 4096:1).

Per another feature of the invention, the power source is a battery, such as, for example, a lithium-polymer battery.

According to another feature, the device may further comprise a controller operative to control operation of the actuator.

The actuator may, per one embodiment, be remotely, wirelessly operable. An RF receiver may, per this feature, be operatively connected to the controller, and the device may be operable by RF signals originating outside of the body of a patient.

Per another embodiment of the invention, the device may further comprise a force sensor for sensing a force applied by the actuator. According to this embodiment, the controller is operatively connected to the force sensor and is further operable to adjust the operation of the actuator in response to the force sensed by the force sensor.

### BRIEF DESCRIPTION OF DRAWINGS

Other advantages of the present invention will be readily appreciated as the same becomes better understood by reference to the following detailed description when considered with the accompanying drawings, wherein:

FIG. 1 is top-down elevational view of an exemplary osteogenesis distraction device according to the present invention;

FIG. 2 is a perspective view of the osteogenesis distraction device of FIG. 1;

FIG. 3 is a lateral, partial cross-sectional view of the device of FIG. 1;

FIG. 4 is an elevational view of the osteogenesis distraction device of the present invention mounted on a mandible; and

FIG. 5 is a schematic illustration of an embodiment of the operation of the present invention.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a representative basis for teaching one skilled in the art to variously employ the present invention.

Referring to FIGS. 1, 2 and 3, an exemplary implantable osteogensis distraction device 10 for securement to separate bone segments in the body of a patient and operative to generate new bone between said bone segments by the gradual application of a distraction force is shown. The device 10 can be seen to generally comprise attachment members 12, 14 securable to separate bone segments and moveable in relation to each other to generate a distraction force, and a power source 18 for powering relative movement of the attachment members.

The entire device, including the attachment members and power source, is dimensioned for implantation entirely within the body of a patient and so may be used in situ, directly in communication and contact with an affected bone structure, to a achieve displacement of the bone for distraction osteogenesis.

The device 10 as shown in the illustrated embodiment more particularly includes a first bone attachment member 12 and a second bone attachment member 14 for securement to separate bone segments. The first bone attachment member 12 and the second bone attachment member 14 define a first axis denoted as X. The first bone attachment member 12 and the second bone attachment member 14 are operatively attached to one another by actuator 16. Power source 18 is operatively connected to the actuator 16. A controller in the form of an electronic control module 20 is in electrical communication with power source 18 and actuator 16. The actuator 16 is adapted to move the second bone attachment member 14 away from the first bone attachment member 12 in a direction along the first axis denoted by X. That is, the actuator 16 provides the distraction force necessary to spatially separate or move the second bone attachment member 14 relative the first bone attachment member 12.

The actuator 16 more particularly includes a direct current (DC) micro-motor 22, a planetary gear head 24 (Faulhaber Micromo Electronics, Clearwater, Fla.) attached to one end thereof having a high reduction ratio (4096:1), and a rotary to linear mechanism having a lead screw 26. The lead screw 26 includes threads which engage a nut 29 attached to a threaded aperture 28 disposed in the second bone attachment member 14 such that when the DC motor 22 is energized, the rotation of the motor 22 through the gear head 24 causes the lead screw 26 to rotate within the nut 29 causing the second bone attachment member 14 to be spatially separated from or to move away from the first bone attachment member 12. The actuator 16 is supported by and rotates within a thrust bearing 25 disposed within the first bone attachment member 12.

Guide pins 30 are disposed within both the first bone attachment member 12 and the second bone attachment member 14 in order to provide support for the second bone attachment member 14 as it is moved away from the first bone attachment member 12, and also to maintain the travel of the second bone attachment member 14 along the desired axis of travel as denoted by X.

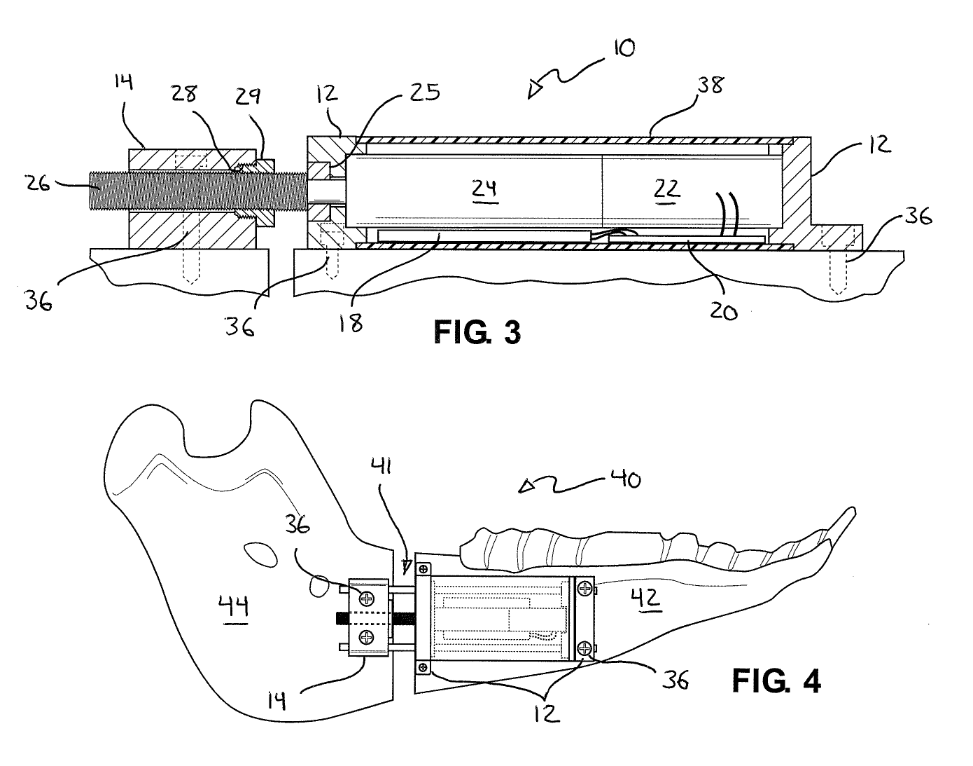

The osteogenesis distraction device 10 further includes apertures 32 disposed within the first bone attachment member 12 for mounting the device 10 to a desired bone fixation/attachment point and apertures 34 are disposed within the second bone attachment member 14 for the same purpose. Screws 36, which are shown in FIGS. 3 and 4, may be used to attach the device 10 to the desired site. However, it is contemplated that other means known in the art for securement of the attachment members to the separate bone segments may also be employed.

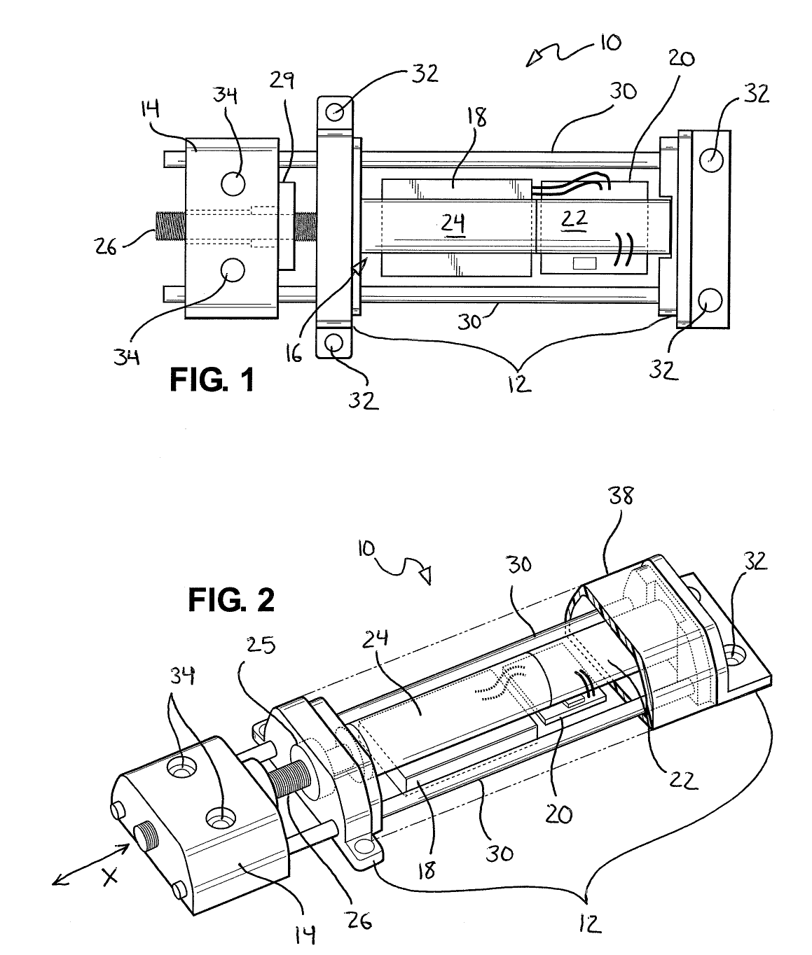

Referring to FIGS. 2 through 4, the device 10 may be encapsulated with a case 38 (not shown in FIG. 1) to enclose all of the device 10 or selected portions such as, for example, the actuator 16, the power source 18, and electronic controller 20.

The DC motor 22 (Faulhaber Micromo Electronics, Clearwater, Fla.) is selected to be utilized in combination with planetary gear head 24 having a high reduction ratio (e.g., 4096:1), and a rotary to linear mechanism by lead screw 26, as this assembly provides the necessary structural stability to transmit sufficient loads with sufficient strain accuracy during distraction osteogenesis. It should be noted that other suitable motors may be utilized in the device 10 of the present invention, as would be readily identifiable by one of ordinary skill in the art.

In an embodiment of the osteogenesis distraction device 10 of the present invention, power source 18 is a battery, such as, by way of example, a lithium-polymer rechargeable battery (UBC322030, Ultra Life Batteries, Newark, N.Y.). Such a battery meets the requirement for a power source having high current discharge. That is, the discharging profile required to perform distraction osteogenesis having a distraction rate of at least 1 mm per day was applied for a total length of 15 mm. The lithium-polymer battery was shown to meet the requirements necessary for performing distraction osteogenesis. The lithium-polymer was able to satisfy the pulse-load profile required by clinical distraction osteogenesis protocols for the necessary duration, i.e., 15 days. This type of battery also demonstrated the necessary performance to cope with prolonged periods of inactivity along with demanding high pulse currents during the distraction period of distraction osteogenesis. The lithium-polymer rechargeable battery was found to satisfy the required high-current discharge (50-70 mA) temperature requirements (i.e., <42.2° C.) and size requirements.

In order to provide the device 10 with the ability to provide continuous distraction force, control of the implantable distraction osteogenesis device 10 of the present invention requires that the speed of the DC motor 22 and the corresponding distraction rate be controlled by controller 20. The controller 20 includes integrated circuit chips, including a clock-counter and a logic gate which can be used to intermittently control the motor speed and the corresponding distraction rate of the device 10. The interval of pulses is dependent upon pin-connections of the clock-counter into the logic gate. Thus, by simply changing the composition of the passive components and their connectivity, the power pulse can be modulated to generate different distraction parameters, such as distraction rate and frequency. Controllers of this type are well known to those of ordinary skill in the art. The controller was assembled using standard surface-mount circuit components on a custom printed circuit board (PCB). The electronic controller 20 can be programmed to perform intermittent or continuous distraction osteogenesis or a combination of both, if desired.

The device 10 can be activated/deactivated to facilitate, cease or stop, respectively, distraction osteogenesis by a variety of mechanisms including, but not limited to, magnetic activation/deactivation of a magnetic switch coupled to the controller 20, radio frequency (RF) activation/deactivation of the controller 20 by the provision of an RF receiver/transmitter operatively connected to the controller 20 and operative to convey data (such as operation instructions, for example) wirelessly received from a remote source, as well as other means well known to those skilled in the art.

Referring to FIG. 4, the implantable osteogenesis distraction device 10 is shown mounted to a portion of a Yucatan minipig mandible 40. The device 10 is affixed to the mandible 40 with the first bone attachment member 12 and second bone attachment member 14 straddling an osteotomy 41 or cut in the bone, which separates a first bone segment 42 from a second bone segment 44. The first bone attachment member is mounted to the first bone segment 42 and the second bone attachment member 14 is mounted to the second bone segment 44.

While the exemplary embodiment of the osteogenesis distraction device comprehends single axis (X) distraction, modifications thereto are possible to allow multi-axial distraction osteogenesis. That is, the device 10 can be configured to allow for distraction osteogenesis in at least two directions or dimensions. In order to do so, the device may in one exemplary modification thereof comprise two or more of the devices 10 of the exemplary embodiment oriented to effect osteogenesis in non-parallel axes.

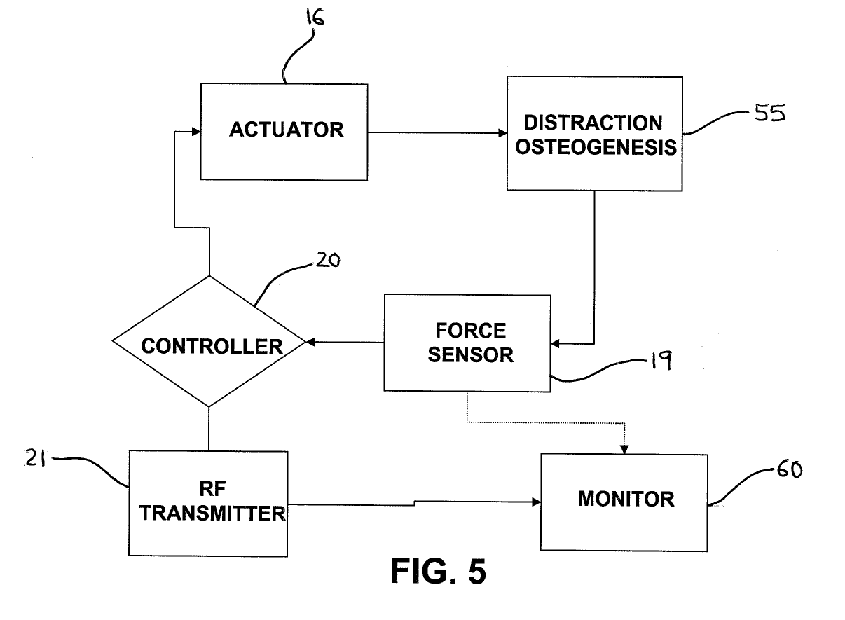

According to one embodiment of the invention, shown in FIG. 5, the device further comprises a force sensor 19 for sensing a force applied by the actuator 16. The force sensor 19 may be powered by power source 18. According to this embodiment, controller 20 is operatively connected to the force sensor 19 and operable to adjust the operation of actuator 16 in response to the force sensed by the force sensor. More particularly, controller 20 is operatively connected to actuator 16 to facilitate distraction osteogenesis leading to the regeneration of tissues. As noted, force sensor 19 senses the distraction force being applied by actuator 16 during the distraction osteogenesis process 55 and this information is provided to controller 20. Controller 20 then adjusts the operation of actuator 16 as necessary to achieve such modifications in the force applied thereby.

The foregoing operation may be effected in a closed-loop operating system, such as shown in FIG. 5, according to which the controller 20 is programmed to automatically make predetermined adjustments to the actuator's operation in response to the force sensed by the force sensor. Alternatively, the operation may be remotely controlled from outside of the patient's body.

An optional radio frequency (RF) transmitter and, optionally, receiver 21, shown in FIG. 5, may be operatively connected to the controller 20, with the controller being programmed to transmit information, such as, for example, the force being applied by the actuator 16, via the RF transmitter to a remote monitor 60, which may, for instance, comprise a computer with a video display, located outside of the patient's body. The RF transmitter could also be operatively connected directly to the force sensor 19 (shown in dashed lines in FIG. 5), and could be programmed to transmit directly to the monitor 60 information from the force sensor 19 respecting the force being applied by the actuator 16 .

Optionally, remote control of the controller 20 could be affected by a user wirelessly conveying to the RF receiver instructions for effecting a change in the actuator's operation via the controller 20 based upon information transmitted to the monitor 60. However, monitoring could also be entirely passive.

The clinical applications and anatomical sites for which the implantable osteogenesis device 10 of the present invention may be utilized includes, but is not limited to, craniomaxillofacial (CAF) such as craniosynostosis; cranium; cleft lip/palate; maxilla; mid-face advancements: maxilla zygoma; frontal bone, orbits; mandibular advancement; mandible; vertical augmentation of alveolar ridges of the maxilla and mandible; sleep apnea; maxilla and mandible; and s/p tumor reconstruction of mandible; transport distraction osteogenesis (used to fill in a continuity defect as opposed to lengthening a bone); and any other bones of the facial skeleton. Orthopedic applications include lengthening of long bones (extremities) such as humorous, ulna, radius, femur, fibula, and tibia. This includes both classical distraction osteogenesis, as well as transport osteogenesis.

As previously stated, the device 10 of the present invention is fully implantable within the subject. No portion of the device 10 is externally disposed or exposed on the subject. Unlike prior distraction osteogenesis devices, once implanted, no portion of the device 10 protrudes through the outside of the subject's body. Distraction osteogenesis is performed without any application of external force.

In operation, the implantable osteogenesis distraction device 10 is implanted within a subject according to the following process. First, access to the site or location for the regeneration of tissues (bone) must be made by a medical/dental practitioner. The medical/dental practitioner then performs an osteotomy in order to sever or divide the bone at the desired site into at least two sections where bone elongation and/or reshaping is desired. The osteogenesis distraction device 10 is then implanted and affixed to the desired location by the application of screws which can be made from titanium or a biodegradable material such as PLA/PGA polymer composite, or the like. The medical/dental practitioner skilled in the relevant art will have knowledge as to the proper location and orientation of the device 10. Proper orientation of the device will also depend upon the desired distraction path and the bone geometry at the attachment site. Following attachment of the device 10 to the desired site, the medical/dental practitioner closes the incision thereby completely disposing the device 10 within the patient. That is, upon completion of the procedure to implant the device 10, no portion of the device is externally disposed on the subject. Following closing of the incision, the device 10 is not immediately activated to begin the distraction osteogenesis process. Rather, the device 10 remains inactive for a predetermined amount of time in order to allow bone cells to begin growing at the site where the bone has been severed. Following this latency period, the device 10 is activated to apply tension between the bone segments and performs distraction osteogenesis at the desired rate for the desired length of time.

The components of the device 10 which come in contact with living tissue, must be formed from non-immunogenic material that is fully biocompatible within the body of the subject which is generally a mammal such as a human, but can also include other animals. Suitable materials include, but are not limited to, titanium, titanium alloys, stainless steel, polycarbonate ISO, and/or other materials known to those skilled in the art. Subsequent to the completion of the distraction osteogenesis in the subject, the device is removed from the subject.

While embodiments of the invention have been illustrated and described, it is not intended that these embodiments illustrate and describe all possible forms of the invention. Rather, the words used in this specification are words of description rather than limitation, and it is understood that various changes may be made without departing from the spirit and scope of the invention

The invention in which an exclusive property or privilege is claimed is defined as follows:

1. An osteogenesis distraction device for securement to separate bone segments in the body of a patient and operative to generate new bone between said bone segments by the gradual application of a distraction force, the device comprising attachment members securable to separate bone segments and moveable in relation to each other to generate a distraction force, a power source for powering relative movement of the attachment members, and wherein the entire device, including the attachment members and power source, is dimensioned for implantation entirely subdermally in the body of a patient.

2. The implantable osteogenesis distraction device of claim 1, wherein relative movement of the attachment members is effected by an actuator operable to generate a distraction force between the attachment members.

3. The implantable osteogenesis distraction device of claim 2, wherein the actuator comprises an electric motor operatively connected to a rotary-to-linear mechanism.

4. The implantable osteogenesis distraction device of claim 3, wherein the rotary-to-linear mechanism is a lead screw operatively connected to one of the attachment members.

5. The implantable osteogenesis distraction device of claim 3, wherein the electric motor is operatively coupled to a transmission.

6. The implantable osteogenesis distraction device of claim 5, wherein the transmission is a planetary gearhead having a high reduction ratio.

7. The implantable osteogenesis distraction device of claim 6, wherein the high reduction ratio is 4096:1.

8. The implantable osteogenesis distraction device of claim 3, wherein the electric motor is a DC micromotor.

9. The implantable osteogenesis distraction device of claim 1, wherein the power source is a battery.

10. The implantable osteogenesis distraction device of claim 9, wherein the battery is lithium-polymer battery.

11. The implantable osteogenesis distraction device of claim 2, further comprising a controller operative to control operation of the actuator.

12. The implantable osteogenesis distraction device of claim 11, wherein the actuator is remotely, wirelessly operable.

13. The implantable osteogenesis distraction device of claim 12, the device further comprising an RF receiver operatively connected to the controller, and wherein further the device is operable by RF signals originating outside of the body of a patient.

14. The implantable osteogenesis distraction device of claim 2, the device further comprising a force sensor for sensing a force applied by the actuator, and the controller being operatively connected to the force sensor and operable to adjust the operation of the actuator in response to the force sensed by the force sensor.

15. A method for effecting osteogenesis in the body of a patient, comprising the steps of:
providing an osteogenesis distraction device for securement to separate bone segments in the body of a patient and operative to generate new bone between said bone segments by the gradual application of a distraction force, the device comprising attachment members securable to separate bone segments and moveable in relation to each other to generate a distraction force, a power source for powering relative movement of the attachment members; and implanting the entire osteogenesis distraction device, including the attachment members and power source, subdermally in the body of a patient.

16. The method for effecting osteogenesis of claim 15, wherein relative movement of the attachment members is effected by an actuator operable to generate a distraction force between the attachment members.

17. The method for effecting osteogenesis of claim 16, further comprising the step of remotely, and wirelessly effecting operation of the actuator after the device is implanted in the body of the patient.
